(19) United States
(12) Patent Application Publication
(10) Pub. No.: US 2017/0360454 A1
(43) Pub. Date: Dec. 21, 2017

(54) MEDICAL HANDPIECE HAVING ATTACHABLE SPRAY, SUCTION AND DRILL UNITS

(71) Applicant: AlloTech Co., LTD., Namyangju-si (KR)

(72) Inventor: Jung Taek KO, Namyangju-si (KR)

(21) Appl. No.: 15/624,787

(22) Filed: Jun. 16, 2017

(30) Foreign Application Priority Data
Jun. 17, 2016 (KR) 10-2016-0075909

(51) Int. Cl. A61B 17/16; A61B 17/00

(52) U.S. Cl. CPC A61B 17/1622; A61B 17/162; A61B 2217/005; A61B 2017/00473

(57) ABSTRACT

Provided is a medical handpiece having liquid spray and suction functions and capable of being replaced with a drill. The medical handpiece includes a handpiece body having an accommodation space therein, a spray unit receiving liquid from an outside to spray the liquid onto a target to be treated, a pump unit transmitting air pressure to the spray unit to increase a spray speed of liquid sprayed by the spray unit, a drive unit converting a rotary motion into a reciprocating motion to transmit power to the pump unit, and a suction unit sucking liquid and foreign matter from the target to be treated.

### CROSS-REFERENCE (S) TO RELATED APPLICATION

This application claims priority of Korean Patent Application No. 10-2016-0075909, filed on Jun. 17, 2016, in the Korean Intellectual Property Office, which is hereby incorporated by reference in its entirety.

### BACKGROUND OF THE INVENTION

### Field of the Invention

The present invention relates to a medical handpiece having liquid spray and suction functions and capable of being replaced with a drill. More particularly, the invention relates to a medical handpiece having liquid spray and suction functions and capable of being replaced with a drill, which has both the function of spraying liquid onto a target to be treated and the function of sucking liquid and foreign matter.

### Description of the Related Art

A cooling-water handpiece and a suction handpiece of a conventional medical handpiece are separately manufactured. Thus, since cooling-water spray and suction are performed by the separate handpieces, a medical staff should hold the respective handpieces to treat a patient. Consequently, this is problematic in that it is inconvenient for the medical staff to perform medical treatment.

### SUMMARY OF THE INVENTION

Accordingly, the present invention has been made keeping in mind the above problems occurring in the related art, and an aspect of the present invention is directed to a medical handpiece having liquid spray and suction functions and capable of being replaced with a drill, in which a handpiece body includes a spray unit functioning to spray liquid onto a target to be treated, and a suction unit sucking liquid and foreign matter from a target to be treated, and a drill unit instead of the spray unit is optionally mounted to drill a hole in the target to be treated.

Objects of the present invention are not limited to the above-mentioned object, and other objects that are not mentioned herein will be clearly understood by those skilled in the art from the following description.

In order to accomplish the object, the present invention provides a medical handpiece having liquid spray and suction functions and capable of being replaced with a drill, including a handpiece body having an accommodation space therein; a spray unit receiving liquid from an outside to spray the liquid onto a target to be treated; a pump unit transmitting air pressure to the spray unit to increase a spray speed of liquid sprayed by the spray unit; a drive unit converting a rotary motion into a reciprocating motion to transmit power to the pump unit; and a suction unit sucking liquid and foreign matter from the target to be treated.

The spray unit may include a longitudinal spray pipe; a spray passage communicating with the spray pipe, and receiving the liquid from the outside to supply the liquid to the spray pipe; and a spray nozzle coupled to an end of the spray pipe.

The drive unit may include a drive motor; a cam part having a connecting rod connected with the drive motor, and an engagement cylinder fixing the connecting rod; a bevel gear part connected with the cam part, and converting a rotary motion of the drive motor into a reciprocating motion; and a drive shaft connected with the bevel gear part, and transmitting power converted into the reciprocating motion to the pump unit.

The bevel gear part may include a first bevel gear coupled with the engagement cylinder to perform a rotary motion; and a second bevel gear engaging with the first bevel gear to rotate in a direction perpendicular to a direction in which the first bevel gear rotates.

The second bevel gear may include a rotating shaft formed on a side of an upper surface of the second bevel gear.

The drive shaft may have on a side thereof a rotary recess, and the rotating shaft may be connected to the rotary recess, so that a reciprocating motion may be performed as the second bevel gear rotates, and thereby power may be transmitted to the pump unit.

The pump unit may include a cylinder having on an outer circumference thereof a bellows pipe; a piston connected with the drive shaft to perform a reciprocating motion in the cylinder; and a compression rod seated on an end of the spray pipe and transmitting air pressure to the spray pipe as the piston may reciprocate.

A drill unit may be optionally coupled to the connecting rod connected with the drive motor, the drill unit including a chuck part coupled with the connecting rod; and a drill bit connected with the chuck part.

The suction unit may include a longitudinal suction pipe; an intake passage sucking liquid and foreign matter that are sucked by the suction pipe; and a suction nozzle coupled to an end of the suction pipe.

The handpiece body may include a trigger to optionally actuate any one of the spray unit, the suction unit and the drill unit.

The medical handpiece having liquid spray and suction functions and capable of being replaced with the drill according to the present invention so as to solve the above-mentioned problems has the following effects.

The present invention is advantageous in that it includes the spray unit functioning to spray liquid onto the target to be treated, and the suction unit sucking liquid and foreign matter from the target to be treated, thus being capable of spraying and sucking liquid only by one body.

Further, the present invention is advantageous in that the drill unit instead of the spray unit may be optionally mounted to drill the hole in the target to be treated.

Effects of the present invention are not limited to the above-mentioned effects, and other effects that are not mentioned herein will be clearly understood by those skilled in the art from claims.

### BRIEF DESCRIPTION OF THE DRAWINGS

The above and other aspects, features and advantages of certain exemplary embodiments of the present invention will be more apparent from the following description taken in conjunction with the accompanying drawings, in which:

FIG. 1 is a view illustrating a medical handpiece having liquid spray and suction functions and capable of being replaced with a drill, according to a first embodiment of the present invention;

FIGS. 2 and 3 are views illustrating sections of the medical handpiece having the liquid spray and suction functions and capable of being replaced with the drill, according to the first embodiment of the present invention;

FIGS. 4A to 4C are views illustrating an operating method illustrating the medical handpiece having the liquid spray and suction functions and capable of being replaced with the drill, according to the first embodiment of the present invention; and

FIG. 5 is a view illustrating a state in which a drill unit of the medical handpiece having the liquid spray and suction functions and capable of being replaced with the drill according to the first embodiment of the present invention is mounted.

### DETAILED DESCRIPTION OF EXEMPLARY EMBODIMENTS

Exemplary embodiments of the present invention will be described below in detail with reference to the accompanying drawings. In the description of the embodiments, the same components will carry the same terms and reference numerals, and a duplicated description thereof will be omitted herein. Further, in the description of the embodiments of the present invention, the configuration of the drawings is merely for the illustrative purpose, and various shapes are possible without limiting the scope of the invention.

The present invention relates to a medical handpiece, and more particularly to a medical handpiece having a spray function of spraying liquid such as cooling water or cleaning water into a target to be treated, and a suction function of sucking liquid of a target to be treated and foreign matter secreted by the body after the liquid is sprayed.

Since heat of high temperature is generated in a target to be treated, for example, skin or bone, in a medical treatment process, it is necessary to spray liquid such as cooling water or cleaning water into the target to be treated so as to cool the heat of high temperature resulting from the treatment process and alleviate a patient's pain, and it is also necessary to suck foreign matter such as blood or pus secreted by the body in the treatment process, namely, suction is required.

Further, according to another embodiment of the present invention, a drill may be optionally mounted to drill a hole in the bone of the body or the like instead of the spray function and the suction function.

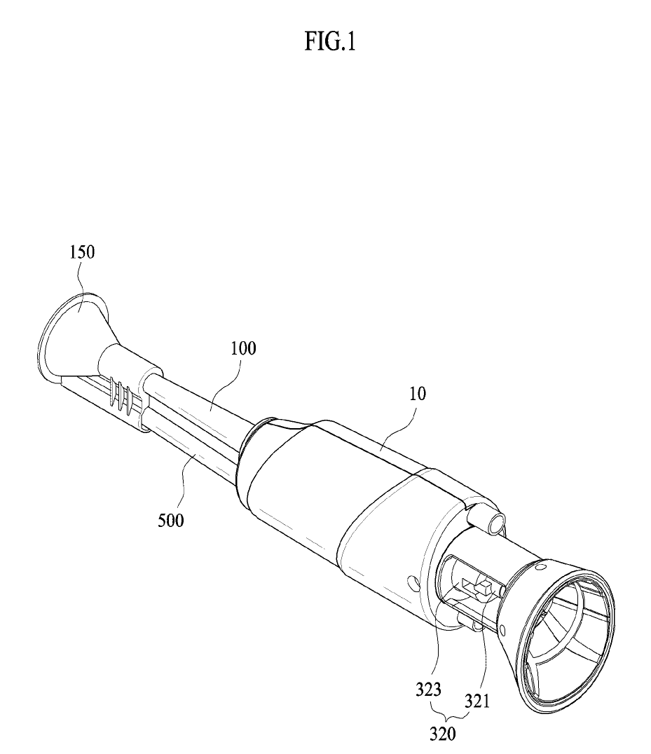

Referring to FIGS. 1 to 3, an accommodation space is defined in the handpiece body 10, and a spray unit 100, a pump unit 200, a drive unit 300 and a suction unit 500 are mounted in the accommodation space.

The spray unit 100 receives liquid, cleaning water or the like from an outside, namely, a separate device (not shown), and functions to spray the liquid onto the target to be treated.

A spray passage 130 receives liquid, cleaning water or the like from the separate device (not shown). Further, a spray pipe 120 communicates with the spray passage 130, and receives the liquid from the spray passage 130, thus spraying the liquid through a spray nozzle 150 coupled to an end of the spray pipe 120.

Although will be described later, the pump unit 200 transmits air pressure to the spray unit 100, thus increasing the spray speed of liquid sprayed by the spray unit 100. Thus, the spray unit 100 sprays the liquid onto the target to be treated, and additionally, the pump unit 200 increases the spray speed of liquid sprayed from the spray unit 100.

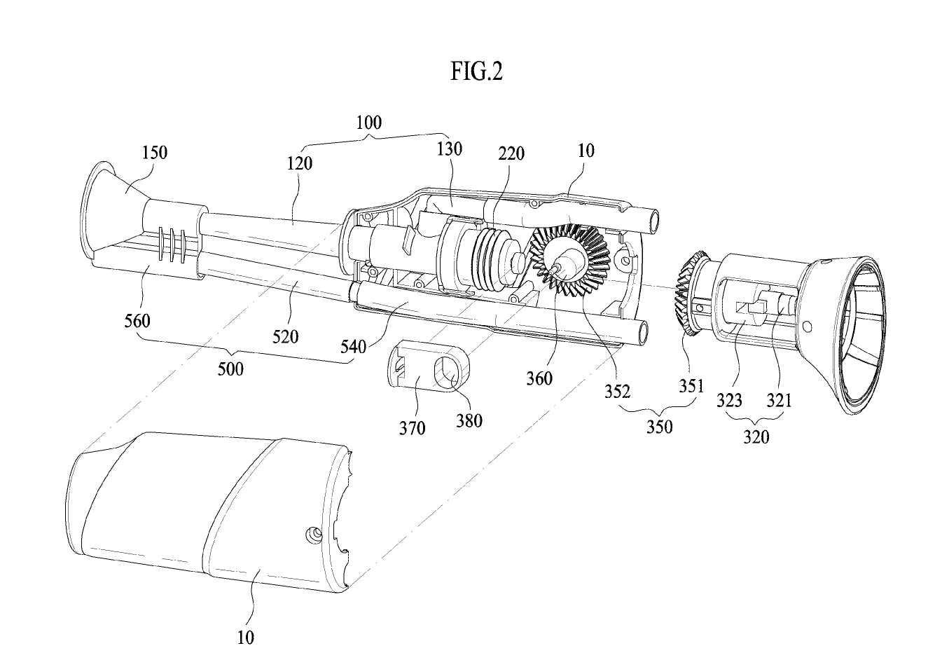

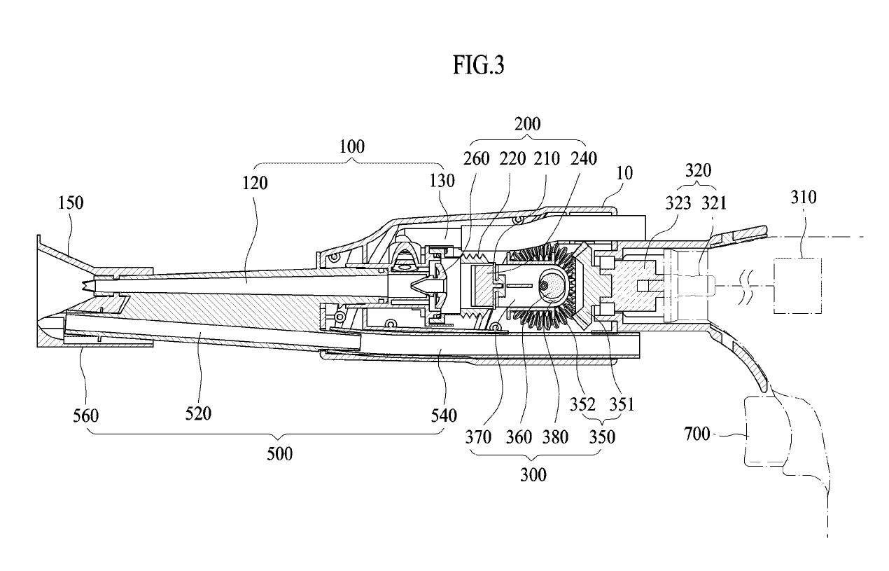

Referring to FIGS. 2 and 3, the suction unit 500 includes a suction pipe, an intake passage 540, and a suction nozzle 560.

The longitudinal suction pipe 520 is provided on a lower portion of the handpiece body 10. Further, the suction nozzle is coupled to an end of the suction pipe 520.

The suction pipe 520 may be separately coupled to the suction nozzle 560, and may be formed integrally with the above-described spray nozzle 150 for the convenience of manufacture.

The suction pipe 520 is connected with the intake passage 540, and the intake passage 540 is connected with a separate intake device (not shown). Thus, if the intake device is actuated, liquid and foreign matter secreted by the body, which are sucked through the suction nozzle 560 and the suction pipe 520, are sucked through the intake passage 540 into the intake device.

Therefore, the medical handpiece according to the present invention has both the liquid spray function and the suction function.

Referring to FIGS. 2 to 4, the drive unit 300 includes a drive motor 310, a cam part 320, a bevel gear part 350, and a drive shaft 370.

The cam part 320 includes a connecting rod 321 connected with the drive motor 310, and an engagement cylinder 323 fixing the connecting rod 321. The connecting rod 321 is detachably coupled to a groove formed in an upper end of the engagement cylinder 323 through interference fitting or the like. The cam part 320 is connected with the bevel gear part 350 that will be described later, and functions to transmit the rotating force of the drive motor 310 to the bevel gear part 350.

The bevel gear part 350 includes a first bevel gear 351 and a second bevel gear 352, and functions to convert the rotary motion of the drive motor 310 into the reciprocating motion.

The first bevel gear 351 is coupled with the engagement cylinder 323. Since all of the drive motor 310, the connecting rod 321 and the engagement cylinder 323 are connected with each other, the rotation of the drive motor 310 leads to the rotation of the first bevel gear 351.

The second bevel gear 352 engages with the first bevel gear 351 to rotate in a direction perpendicular to the rotating direction of the first bevel gear 351.

A cylindrical rotating shaft 360 is provided on a side of an upper surface of the second bevel gear 352. That is, the rotating shaft 360 is not located at a center of the upper surface of the second bevel gear 352 but is located eccentrically.

The drive shaft 370 has on a side thereof a rotary recess 380. As the rotating shaft 360 of the second bevel gear 352 is inserted into the rotary recess 380 and then the second bevel gear 352 rotates, the reciprocating motion is performed and thereby power is transmitted to the pump unit 200 that will be described later.

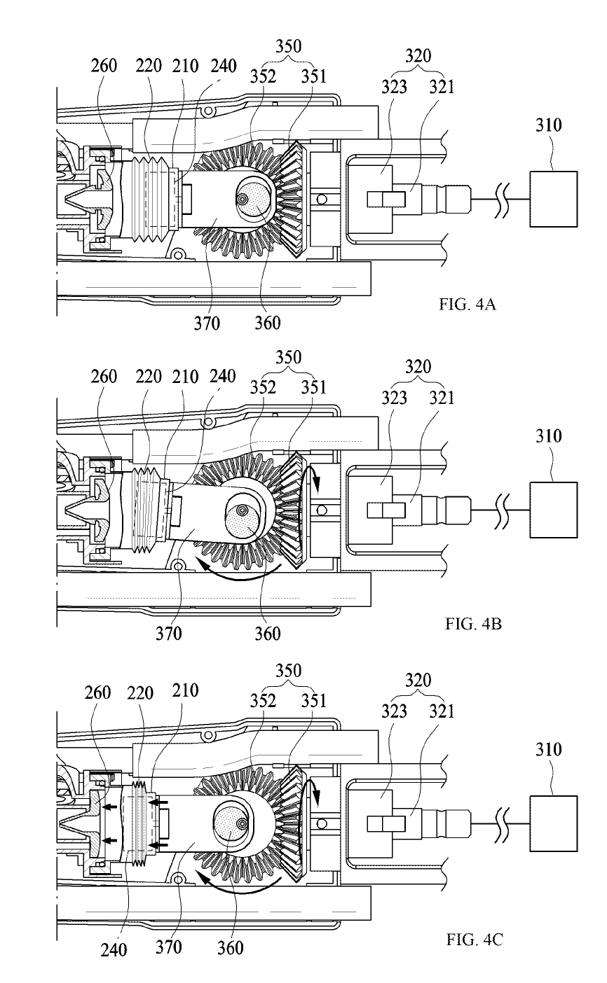

Referring to the embodiment illustrated in FIGS. 3, 4A to 4C, when the rotating shaft 360 is located on a right side of the upper surface of the second bevel gear 352 (FIG. 4A), the drive shaft 370 is located on the right side with respect to the upper surface of the second bevel gear 352.

If the drive motor 310 and the first bevel gear 351 rotate, the second bevel gear 352 rotates clockwise, and the drive shaft 370 is located at a lower position with respect to the upper surface of the second bevel gear 352 together with the rotating shaft 360 (FIG. 4B). In this case, the drive shaft 370 is slightly inclined.

Further, if the drive motor 310 and the first bevel gear 351 continue to rotate, the drive shaft 370 is located on a left side with respect to the upper surface of the second bevel gear 352 together with the rotating shaft 360 (FIG. 4C).

As such, as the second bevel gear 352 rotates, the drive shaft 370 performs the reciprocating motion. More precisely, the right side on which the rotary recess 380 of the drive shaft 370 is formed performs the rotary motion, and the left side of the drive shaft 370 performs the reciprocating motion while being inclined.

Referring to FIGS. 3, 4A to 4C, the pump unit 200 includes a cylinder 210, a piston 240, and a compression rod 260.

The piston 240 is installed in the cylinder 210. The piston 240 is connected with the drive shaft 370 to reciprocate in the cylinder 210.

Further, a bellows pipe 220 is formed on an outer circumference of the cylinder 210. The reason why this configuration is adopted is because the drive shaft 370 should perform the reciprocating motion while being inclined, and the piston 240 and the cylinder 210 connected with the drive shaft 370 also should be rotatable up and down while being inclined.

The compression rod 260 is seated on an end of the spray pipe 120, thus functioning to transmit air pressure to the spray pipe 120 as the piston 240 reciprocates.

An upper portion of the compression rod 260 has the shape of a gentle arc, and a lower portion thereof has a longitudinally extending shape. The lower portion of the compression rod 260 is inserted into an end of the spray pipe 120 to be slightly spaced apart therefrom.

The compression rod 260 is preferably made of soft rubber or the like to compress the upper portion of the compression rod 260 and transmit a compressive force to the end of the spray pipe 120, in the case of increasing air pressure in the cylinder 210 while the piston 240 reciprocates in the cylinder 210.

That is, as illustrated in FIG. 4C, when the second bevel gear 352 rotates and thereby the piston 240 compresses air in the cylinder 210, the upper portion of the compression rod 260 is compressed and simultaneously the lower portion of the compression rod 260 inserted into the spray pipe 120 to be slightly spaced apart therefrom comes into close contact with the spray pipe 120, thus further increasing the spray speed of the liquid when the liquid introduced into the spray pipe 120 of the spray unit 100 is sprayed.

Hereinafter, the operating method of the medical handpiece having the liquid spray and suction functions and capable of being replaced with the drill according to the present invention will be described.

Referring to FIG. 2, FIG. 3 and FIGS. 4A to 4C, the spray unit 100 receives liquid, cleaning water or the like to spray the liquid through the spray pipe 120 and the spray nozzle 150 onto the target to be treated.

Further, when the rotating shaft 360 is located on the right side of the second bevel gear 352 (FIG. 4A), the drive shaft 370 is located on the right side and the piston 240 connected with the drive shaft 370 is located at an upper position in the cylinder 210. In this case, air in the cylinder 210 is not compressed.

If the drive motor 310 and the first bevel gear 351 rotate, the second bevel gear 352 rotates clockwise, the drive shaft 370 is slightly inclined, and the piston 240 and the cylinder 210 connected with the drive shaft 370 rotates downwards while being inclined (see FIG. 4B).

Further, if the drive motor 310 and the first bevel gear 351 continue to rotate, the drive shaft 370 is located on the left side, and the piston 240 connected with the drive shaft 370 compresses air in the cylinder 210 while being located at a lower position in the cylinder 210 (see FIG. 4C).

Then, the upper portion of the compression rod 260 is compressed and simultaneously the lower portion of the compression rod 260 inserted into the spray pipe 120 to be slightly spaced apart therefrom comes into close contact with the spray pipe 120, thus further increasing the spray speed of liquid when the spray unit 100 sprays the liquid onto the target to be treated.

This will describe a process in which the pump unit 200 is operated while the bevel gears 351 and 352 rotate, in stages. In the case of operating the handpiece according to the present invention, the bevel gears 351 and 352 rotate at high speed. Consequently, while the spray unit 100 sprays the liquid, the pump unit 200 is also operated at high speed, thus increasing the spray speed of the liquid that is sprayed from the spray unit 100.

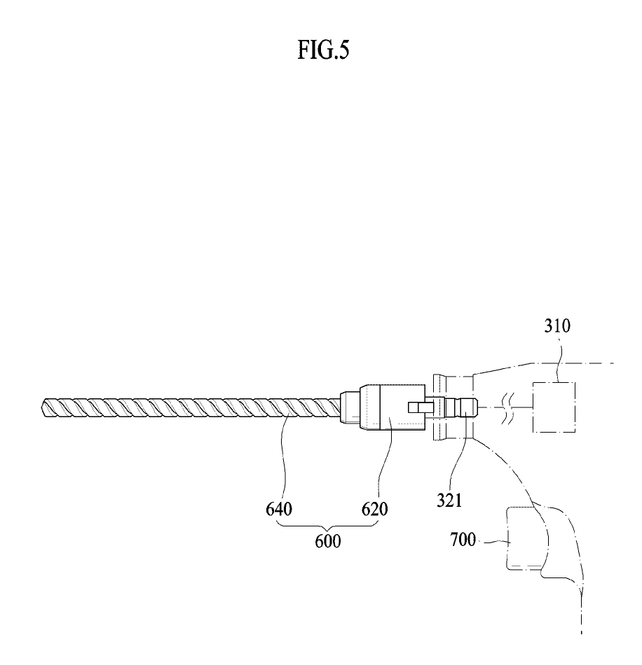

Referring to FIGS. 3 to 5, a drill unit 600 may be optionally mounted to the medical handpiece according to the present invention.

The drill unit 600 includes a chuck part 620 and a drill bit 640.

Various kinds of drill bits 640 used in bone surgery or the like may be mounted. The drill bit 640 is connected with the chuck part 620. The chuck part 620 means a component for fixing the drill to a drilling machine or the like.

Further, the chuck part 620 is optionally coupled with the connecting rod 321 that is connected to the drive motor 310. A replacement process with the drill is as follows.

First, the handpiece body 10, the spray unit 100, the pump unit 200 and the bevel gear part 350 are separated. After the engagement cylinder 323 is separated from the connecting rod 321, the chuck part 620 of the drill unit 600 is coupled to the connecting rod 321 connected to the drive motor 310. Thus, the medical handpiece having the liquid spray and suction functions and capable of being replaced with the drill according to the present invention may be used as a drill that is employed to perform an operation on the bone of the body or the like.

Referring to FIGS. 3 to 5, the handpiece body 10 includes a trigger 700 to optionally actuate any one of the spray unit 100, the suction unit 500 and the drill unit 600. Further, the handpiece body 10 includes a controller (not shown) that is connected with the trigger 700 to control the operation of the spray unit 100, the suction unit 500, and the drill unit 600 by pulling the trigger 700.

The embodiment of the present invention may be configured such that the spray unit 100 is operated by pulling the trigger 700 once and the suction unit 500 is operated by pulling the trigger 700 once more.

Further, even in the case of mounting the drill unit 600 on the medical handpiece according to the present invention, the drill unit 600 may be actuated by the trigger 700, as in the above-described embodiment.

While the invention has been shown and described with reference to exemplary embodiments thereof, it will be understood by those skilled in the art that various changes in form and details may be made therein without departing from the spirit and scope of the invention as defined by the appended claims. Therefore, it is to be understood that the scope of the invention is not limited to the above-described embodiments, and all changes that fall within meets and bounds of the claims, or equivalence of such meets and bounds are intended to be embraced by the claims.

1. A medical handpiece having liquid spray and suction functions and capable of being replaced with a drill, comprising:
a handpiece body having an accommodation space therein;
a spray unit receiving liquid from an outside to spray the liquid onto a target to be treated;
a pump unit transmitting air pressure to the spray unit to increase a spray speed of liquid sprayed by the spray unit;
a drive unit converting a rotary motion into a reciprocating motion to transmit power to the pump unit; and
a suction unit sucking liquid and foreign matter from the target to be treated.

2. The medical handpiece of claim 1, wherein the spray unit comprises:
a longitudinal spray pipe;
a spray passage communicating with the spray pipe, and receiving the liquid from the outside to supply the liquid to the spray pipe; and
a spray nozzle coupled to an end of the spray pipe.

3. The medical handpiece of claim 2, wherein the drive unit comprises:
a drive motor;
a cam part having a connecting rod connected with the drive motor, and an engagement cylinder fixing the connecting rod;
a bevel gear part connected with the cam part, and converting a rotary motion of the drive motor into a reciprocating motion; and
a drive shaft connected with the bevel gear part, and transmitting power converted into the reciprocating motion to the pump unit.

4. The medical handpiece of claim 3, wherein the bevel gear part comprises:
a first bevel gear coupled with the engagement cylinder to perform a rotary motion; and
a second bevel gear engaging with the first bevel gear to rotate in a direction perpendicular to a direction in which the first bevel gear rotates.

5. The medical handpiece of claim 4, wherein the second bevel gear comprises a rotating shaft formed on a side of an upper surface of the second bevel gear.

6. The medical handpiece of claim 5, wherein the drive shaft has on a side thereof a rotary recess, and the rotating shaft is connected to the rotary recess, so that a reciprocating motion is performed as the second bevel gear rotates, and thereby power is transmitted to the pump unit.

7. The medical handpiece of claim 6, wherein the pump unit comprises:
a cylinder having on an outer circumference thereof a bellows pipe;
a piston connected with the drive shaft to perform a reciprocating motion in the cylinder; and
a compression rod seated on an end of the spray pipe, and transmitting air pressure to the spray pipe as the piston reciprocates.

8. The medical handpiece of claim 3, wherein a drill unit is optionally coupled to the connecting rod connected with the drive motor, the drill unit comprising:
a chuck part coupled with the connecting rod; and
a drill bit connected with the chuck part.

9. The medical handpiece of claim 1, wherein the suction unit comprises:
a longitudinal suction pipe;
an intake passage sucking liquid and foreign matter that are sucked by the suction pipe; and
a suction nozzle coupled to an end of the suction pipe.

10. The medical handpiece of claim 1, wherein the handpiece body comprises a trigger to optionally actuate any one of the spray unit, the suction unit and the drill unit.
